(19) United States
(12) Patent Application Publication
(10) Pub. No.: US 2010/0221788 A1
(43) Pub. Date: Sep. 2, 2010

(54) METHOD FOR RECOVERING SHORT RNA, AND KIT THEREFOR

(76) Inventors: Edmund RADMACHER, Duren (DE); Klaus Moller, Eschweiler (DE)

Correspondence Address: BERENATO & WHITE, LLC, 6550 ROCK SPRING DRIVE, SUITE 240, BETHESDA, MD, 20817, US

(21) Appl. No.: 12/548,830

(22) Filed: Aug. 27, 2009

(30) Foreign Application Priority Data
Sep. 4, 2008 (DE) 102008045705.1-41

(51) Int. Cl. C12P 19/34; C07H 21/04; C12N 9/16

(52) U.S. Cl. 435/91.5; 536/25.41; 435/196

(57) ABSTRACT

The invention relates to a method for recovering at least short RNA, having at least the following steps:
a) making available a biological solution containing at least short RNA as well as proteins and/or long nucleic acids (long RNA and DNA);
b) removing the proteins and the long nucleic acids from the solution, at least the proteins being precipitated;
c) adsorbing the short RNA onto a solid (first) carrier after precipitation of the proteins;
d) recovering the short RNA by desorption from the carrier.

The invention further includes a kit for carrying out the method.

### CROSS-REFERENCE TO RELATED APPLICATIONS AND CLAIM TO PRIORITY

This application is related to application no. 10 2008 045705.1-41, filed Sep. 4, 2008 in the Federal Republic of Germany, the disclosure of which is incorporated by reference and to which priority is claimed.

### FIELD OF THE INVENTION

The present invention relates to a multi-step method for recovering at least short RNA, and to a kit therefor.

### BACKGROUND OF THE INVENTION

It has been become evident in recent years that short RNA in cells are not unimportant breakdown products of long RNA, for example of rRNA or mRNA, but rather that they are specifically synthesized. They play an essential role in gene regulation, RNA synthesis, RNA modification, and RNA splicing in almost all living organisms, and new functions (and therefore new areas of application) are continually being discovered. In accordance with their function, structure, location in the cell, or reaction partners, these small RNAs are subdivided into groups such as, for example, miRNA, siRNA, shRNA, snoRNA, scnRNA, piRNA, tasiRNA, rasiRNA, etc., the number of which is likewise constantly increasing.

The short RNAs occur mostly in single-stranded form, and typically have lengths in the range from 17 to 35 nucleotides. There are, however, also short double-stranded RNAs such as, for example, siRNA or shRNA, although these are also converted into single strands in order to exert their effect.

Whereas the biosynthesis and function of most short RNAs have not yet been completely deduced, it has already been possible to develop, for some species, concrete applications of economic and scientific interest. In this context, miRNA and siRNA are of particular interest.

MiRNAs (microRNAs) are nucleus-coded, are synthesized in the cell, and generally result, by way of various mechanisms, in targeted repression of gene expression. The relevant genes are chiefly those participating in the development, division, and differentiation of cells. It has been shown that the miRNA expression profile of healthy cells differs considerably from what is found in cells with disrupted growth, e.g. in the context of cancer. Using chipbased “miRNA profiling,” i.e. quantification of a set of miRNAs, it is now possible to identify a (cancer-related) disease much more accurately than with previous methods.

The formation of double-stranded siRNA (short interfering RNA) is utilized in almost all plant and animal cells for defense against pathogens. After a pathogen penetrates into the cell, intermediate formation of double-stranded RNA usually occurs, this being cut up, by Dicer enzyme complexes endogenous to the cell, into small siRNA. The siRNA itself then serves as a sequence-specific probe for the mRNA that is foreign to the cell, which is thereby identified, cut, and thereby rendered harmless by special nuclease complexes (RISC). This repression at the mRNA level can thus be used to restrict the expression of any genes (including those endogenous to the cell), and therefore principally the corresponding proteins. This is done, for example, by introducing in-vitro synthesized siRNA into the cells, or else intracellular siRNA synthesis is provided via vector systems. The short RNAs in the cells cause the target genes, which are defined by the sequence of the short RNAs, to be repressed by way of the mechanisms described above. This opens up a very elegant and simple capability for specifically inactivating individual genes, which is very valuable for the investigation of gene regulation and protein function. This method is thus greatly superior to conventional methods, for example the production of knock-out mutants, in terms of cost, time expended, and general feasibility.

For the aforesaid applications such as transfection with siRNA or miRNA profiling, but also for further deciphering of structure and functional mechanisms, it is necessary to purify the active RNA species from the sample materials in as quantitative a fashion as possible. In addition, for subsequent analysis that is becoming more and more sensitive, a very large background of long RNA, DNA, and protein must be removed from the short RNA. This places very high demands on the extraction method, since the short RNAs are often present only in trace amounts.

According to the present existing art, almost every purification of short RNA is based on the principle of firstly removing the large macromolecules, such as DNA, long RNA, and in some cases also protein, from the lysate of a biological sample. The small RNA is then precipitated with alcohol and then isolated usually by binding to a solid carrier. The critical aspect of this method is the removal of protein as completely as possible before precipitation of the small RNA, since the protein otherwise co-precipitates and makes further purification of the small RNA very difficult.

WO 2005/012523 A1 describes a method for obtaining short RNA having 100 or fewer nucleotides, in which method the removal of DNA and protein is carried out by liquid-liquid extraction with phenol/chloroform. After extraction, all the RNA is located in the aqueous phase, which must then be separated from the organic phase containing the majority of the proteins, and from the DNA-containing interphase. The addition of alcohol to the aqueous phase then creates a condition under which the RNA binds to a solid carrier.

A disadvantage of this method is that the use of phenol entails a considerable health risk to the user, since phenol is toxic and corrosive. It may also prove difficult to remove the aqueous phase quantitatively after liquid-liquid extraction without thereby carrying over traces of the interphase having DNA, or of the phenolic phase having protein. If, for this reason, the aqueous phase is not completely removed, considerable decreases in RNA yield must be accepted. A further disadvantage of this method can be the fact that liquid-liquid extraction, and the phase separation associated therewith, cannot readily be automated. Especially with regard to possible future routine diagnosis by means of miRNA profiling, the complex and tiresome procedure and the limitation on sample throughput imposed by manual processing represent a serious disadvantage.

Other methods therefore dispense with extraction using organic solvents. WO 2005/012487 A2, for example, proposes a method in which long RNA and DNA are separated by column chromatography in the presence of small quantities of alcohol. The small RNA remains in solution, and is bound to a second carrier after the alcohol concentration is raised.

A disadvantage of this method is that proteins that are also present in the solution are not completely separated out by the method. When the alcohol concentration is raised in order to separate out the small RNA, the proteins can co-precipitate as described above, and can bind to the carrier along with the small RNA. It then becomes almost impossible to wash out the protein completely. A further disadvantage results from the fact that the cellular proteins often also include RNases that, without quantitative purification, digest the isolated RNAs and thus lower the yield or contaminate it with breakdown products, or in the worst case make it unusable.

A further method for purifying small RNA species is described in US 2007/0202511 A1. Simultaneous or sequential addition to an RNA-containing solution of a chaotropic reagent and metal salts of the first and second main groups of the periodic table causes long RNAs and genomic DNA to precipitate, while short RNA molecules remain in solution. The supernatant containing the short RNA is then separated from the precipitate, and the short RNAs are further purified. Centrifuging methods, or a variety of chromatographic methods, are used for this.

A disadvantage of this method is that in this case as well, complete protein removal is not guaranteed. The method requires subsequent chromatographic purification of the small RNA, for which once again precipitation with alcohol—whose problem in the present of protein has already been explained—is proposed. No provision is made for specifically separating the proteins.

A method that results in removal of the protein, but makes do without liquid-liquid extraction using phenol, is found in the commercial product “AllPrep DNA/RNA/Protein” of the Qiagen company, Hilden (DE), for the purification of DNA, long RNA, and protein. Here DNA is bound to a first silica membrane in the present of chaotropic salt. After the addition of alcohol, the RNA can then also be bound to a second silica membrane. The remaining lysate has a zinc-containing solution added to it in order to recover the protein. The protein is precipitated in this context, and can be isolated by centrifuging.

This method is not intended, or suitable, for the purification of short RNA, since the biochemistry used in the proposed kit results in a cutoff of approx. 200 nucleotides in interplay with silica membranes, so that short RNA is lost in the course of purification.

### SUMMARY OF THE INVENTION

It is the object of the present invention to create a method that enables the recovery, in as quantitative a manner as possible, of short RNA from a sample, and simultaneously dispenses very largely with toxic chemicals. In addition, the method should be suitable for being carried out in an automated method, i.e. without the fluid-fluid-extraction, e.g. with phenol/chloroform, which is difficult to automate. The short RNA that is obtained should also have a high degree of purity. In addition, the method should create the capability of separately recovering the further constituents of such a sample, in particular long nucleic acids and proteins; these, too, should be obtained in as quantitative a fashion as possible and at high purity. A further object is that of making available a kit suitable for carrying out the method.

The first part of the object is achieved, according to the present invention, by a method having the following steps:
a) making available a biological solution containing at least short RNA as well as proteins and/or long nucleic acids (long RNA and DNA);
b) removing the proteins and the long nucleic acids from the solution, at least the proteins being precipitated;
c) adsorbing the short RNA onto a solid (first) carrier after precipitation of the proteins;
d) recovering the short RNA by desorption from the carrier.

It has been found, surprisingly, that in a method in which at least initially the proteins are precipitated from the RNA-containing solution, and the short RNA is then adsorbed onto a carrier from which it can be desorbed again in a further step, the short RNA that is contained is obtained at high yield simultaneously with high purity.

If larger quantities of proteins were still present at the point in time at which the short RNA was adsorbed onto the first carrier, they would likewise precipitate under the corresponding conditions and would bind to the binding matrix. In the worst case, clogging of the binding matrix would occur, as well as considerable contamination of the short RNA with protein. In accordance with the method according to the present invention, protein carryover and/or protein precipitation of this kind is almost precluded by the previous precipitation of the protein, and subsequent washing steps can be reduced to a minimum.

A further advantage of the method according to the present invention is the capability of recovering proteins in a form such that they are usable directly for further purposes. This is not the case with all the other methods for recovering short RNAs.

“Short RNA” is understood, in the context of the present invention, as RNA molecules that typically comprise up to 200 nucleotides, in particular up to 150 nucleotides or up to 100 nucleotides or even up to only 75 nucleotides or only 40 nucleotides. The short RNA is to be separated in particular, by way of the method according to the present invention, from long RNA, DNA, and proteins.

In the context of this invention, short RNA differs from long RNAs such as, for example, ribosomal RNA (rRNA) or messenger RNA (mRNA). Small RNAs for purposes of this invention are, for example, 5.8S rRNA, 5S rRNA, transfer RNAs (tRNA), small nuclear RNA (snRNA), small nucleolar RNA (snoRNA), micro RNA (miRNA), small interfering RNA (siRNA), trans-acting siRNA (tasiRNA), repeat-associated siRNA (rasiRNA), small temporary RNA (stRNA), tiny non-coding RNA (tncRNA), small scan RNA (scRNA), and small modulatory RNA (smRNA).

Natural biological materials such as cells, tissue, organs, organisms, body fluids, etc. can serve as a source from the short RNAs to be extracted, as can in vitro reaction mixtures in which RNA molecules are produced, cut, or modified, for example an in vitro digestion of long dsRNA by Dicer. The method according to the present invention is applicable in principle to all samples containing small RNA.

In order to make the biological solution available, the samples are firstly subjected to a lysis. It is usual to use lysis buffer having chaotropic reagents for this purpose. Chaotropic reagents are known to the skilled artisan. They destroy membranes, denature proteins, result in cell lysis, and release nucleic acids. Ions are arranged in the so-called Hofineister series in accordance with their tendency to attenuate hydrophobic interactions. Ions having a pronounced chaotropic character are, for example, guanidinium, barium, and calcium. Chaotropic salts frequently used in nucleic acid purification are, among others, guanidinium hydrochloride, guanidinium thiocyanate, or sodium perchlorate. The above-described salts result, in the context of lysis, in dissolution of cells or cell assemblages, while simultaneously inactivating RNases. Lysis can furthermore be mechanically assisted in accordance with the existing art. The skilled artisan can call upon a plurality of capabilities for this purpose, for example rotor/stator systems, mortars, ball mills, etc. Lysis is usually followed by processes in which insoluble (cell) constituents are removed, for example, by filtration or centrifuging.

Precipitation of the proteins from the solution is accomplished prior to adsorption of the short RNA on the solid carrier. The long nucleic acids can also be removed along with precipitation of the proteins. It is recommended, however, especially if the long nucleic acids are to be recovered for additional purposes, to adsorb the long nucleic acids on a solid (second) carrier, preferably before precipitation of the proteins. This enables almost complete separation of the long nucleic acids, and the possibility also exists of sending them on separately from the proteins for further utilization. For this, the long nucleic acids are desorbed, in particular eluted, from the solid (second) carrier, optionally after at least one washing operation.

The method makes it possible for the first time to isolate protein, long RNA, and short RNA from a single, undivided sample at high purity and, most of all, also quantitatively. The method thereby opens up the possibility of reliable quantitative determination of short RNA but also of long nucleic acids and proteins. This is a critical advance in terms of the investigation of gene expression, since the quantity of many small RNAs (e.g. miRNA) has a regulatory influence on the quantity of many mRNAs (long RNA), which in turn contributes to determining the quantity of protein respectively expressed. A exact knowledge of the quantities of the three components that are present is important, however, not only in terms of the investigation of natural regulation processes. The mRNA and protein content can be regulated by means of small RNAs (e.g. siRNA) introduced from outside into the cell. Exact investigation of a dose-effect relationship in turn requires a knowledge of the quantities of small RNA, long RNA, and protein present in a sample.

An particularly water-miscible organic solvent should be added to the lysate in a quantity that is sufficient to bind long nucleic acids (RNA and DNA) to the carrier. In preferred fashion, the solvent concentration is adjusted to 15 to 40%, particularly preferably to 20 to 30%, even better to 25%, based in each case on the solution as a whole. These concentrations result in good adsorption of the long nucleic acids, but leave the small RNA in the solution. Preferred organic solvents are one or more alcohols, in particular ethanol and/or 2-propanol.

A further embodiment of the method according to the present invention provides for adding a salt at high concentration to the solution for adsorption of the long nucleic acids. A chaotropic salt, or a mixture of multiple chaotropic salts, is particularly suitable for this. The term “high concentration” is understood in this context as salt concentrations greater than or equal to 1 M. It is particularly advantageous if the concentration of the salt in the solution is adjusted to a range from 1 to 10 M.

If long RNA is to be recovered in isolated fashion, it is useful to remove the DNA from the nucleic acids adsorbed on the second carrier. This can be accomplished, for example, by DNase digestion. The long RNA can then be eluted from the second carrier, optionally after a washing operation using suitable washing buffers.

For removal of the proteins, the solution that contains the short, unbound RNA species has a protein-precipitating reagent added to it. The proteins are preferably precipitated using a solution containing divalent metal ions. Salts of the corresponding metals are used for this. Suitable metals in this context are, for example, Fe, Co, Ni, Cu, Zn, Cd, Hg, Pb, and Ba, usefully in a concentration range (effective final concentration) from 0.01 to 1.5 M, better 0.05 to 1 M, even better 0.1 to 0.8 M, particularly preferably 0.2 to 0.6 M, and optimally 0.30 to 0.40 M.

The precipitated proteins are then removed from the solution by centrifuging or by passage through a suitable filter apparatus. The throughput is then largely free of protein. The separated-out protein is available directly for further analyses. In known fashion, for example, it can be dissolved in Laemmli buffer, quantified, subjected to gel electrophoresis, and used in western blots. It has been ascertained, surprisingly, that this protein precipitation, which is also suitable for precipitating long RNA or DNA and therefore was used hitherto only after the removal of RNA and DNA, does not affect the small RNA species.

Adsorption of the short RNA onto a solid (first) carrier after precipitation of the proteins is promoted or achieved by the fact that further organic solvents miscible with water are added to the solution until a high concentration is reached, preferably until achieving a final concentration of 30 to 80 vol %, by preference 40 to 70 vol %, in particular approximately 50 to 60 vol %, based in each case on the entire solution.

Particularly suitable solvents in this case are nonalcoholic solvents. Representatives of this group are, for example, acetone, acetonitrile, dimethyl sulfoxide (DMSO), tetrahydrofuran (THF), dioxan, and dimethylformamide (DMF). These can be used individually or as a mixture with one another. These solvents make possible good adsorption of the short RNA on the carrier. A further advantage of the use of nonalcoholic organic solvents for precipitation of the short RNA is that a reduction in contamination of the short RNA with proteins can be achieved in this fashion.

In order to recover the short RNA by desorption from the carrier, the carrier is washed and the bound short RNA molecules are then eluted in highly pure, concentrated form.

A “solid carrier” is understood as solid phases that are water-insoluble and bind to the nucleic acids in the aqueous phase preferably with high ionic strength. Examples are porous or nonporous particles such as silica, glass, quartz, zeolites, or mixtures thereof. The term “solid phase” can furthermore refer to magnetic or nonmagnetic particles, polymer and materials that are coated, for example, with silica, glass, quartz, or zeolites. The solid carrier can furthermore be present in the form of powders or suspensions, or else can be embodied in the form of a membrane, a filter layer, a frit, a monolith, or a solid body of some other kind. In the context of the method according to the present invention and the kit according to the present invention, the first and second solid carrier can be identical or different.

Silica membranes or nonwoven glass-fiber fabrics are preferably used as solid carriers. When solid carriers in the form of membranes or nonwoven fabrics are used, flow through these binding matrices can be effected by gravitation, centrifuging, or application of a vacuum.

A particular embodiment of the method according to the present invention provides for immobilizing the RNA binding matrix, in one or more layers, in hollow elements having an inlet and outlet opening. Hollow bodies of this kind are known to the skilled artisan, for example, as MiniSpin centrifuge elements. Alternatively, however, the binding matrix can also be present in the form of magnetic or nonmagnetic particles. Whereas in the case of the MiniSpin columns the binding, washing, separating, and eluting steps are effected by centrifugal force, magnetic separation can be employed when magnetic beads are used. Corresponding apparatuses are known to the skilled artisan. If nonmagnetic beads are used, separation can then be brought about by sedimentation or centrifuging.

With particulate solid carriers, incubation of the lysate with the binding matrix is performed, followed by separation of the solid carrier. This can be accomplished, for example, as described above, by sedimentation, centrifuging, or filtration. This applies to both first and second solid carriers in the context of the method according to the present invention.

A further subject of the present invention is a kit for carrying out the method according to the present invention, containing
a) at least one solid carrier for the adsorption of nucleic acids;
b) a protein-precipitating reagent;
c) at least one binding substance for selective adsorption of short RNA on a solid carrier;
e) instructions having a description of the method steps of the method according to the present invention.

With the aid of the kit according to the present invention, the user is given, in addition to an assemblage of all the chemicals and material necessary for carrying out the method according to the present invention, instructions that reduce the risk of utilization errors, so that success in practical execution of the method according to the present invention can thereby be ensured. The instructions according feature e) can be provided for example in written form or stored on a CD or DVD.

According to a preferred embodiment, at least one binding substance for adjusting the binding conditions for selective adsorption of long nucleic acids on a solid carrier is present in the kit according to the present invention. Suitable as a binding substance are organic solvents described in further detail, in particular a water-miscible, organic solvent that encompasses one or more alcohols, principally ethanol and/or propanol.

In a further embodiment, a salt made up of one or more chaotropic salts is present in the kit according to the present invention as at least one binding substance. The kit can further comprise a DNase for DNA digestion.

According to a further preferred embodiment, the kit comprises as protein precipitating reagent at least one compound containing metal ions. These are, as a rule, metal salts. The compound containing metal ions is preferably selected from compounds or salts that contain the elements Fe, Co, Ni, Cu, Zn, Cd, Hg, Pb, and/or Ba.

A further assemblage of the kit according to the present invention is notable for the fact that an organic solvent is present as at least one binding substance for the adsorption of short RNA. This is preferably a water-miscible nonalcoholic solvent, in particular acetone, acetonitrile, dimethyl sulfoxide (DMSO), tetrahydrofuran (THF), dioxan, and dimethylformamide (DMF), or mixtures thereof.

For the kit according to the invention, solid carriers are in consideration, which are suitable for the adsorption of nucleic acids.

The kit can furthermore contain at least washing buffer, as well as at least one elution reagent for desorption of at least the short RNA from the carrier.

### DESCRIPTION OF THE FIGURES

Preferred embodiments of the present invention will now be described individually with reference to exemplifying embodiments in combination with pertinent Figures, in which:

FIG. 1 is an electropherogram of a long-RNA fraction from porcine liver, purified in accordance with the method according to the present invention on silica membrane columns;

FIG. 2 is an electropherogram of a short-RNA fraction from porcine liver, purified in accordance with the method according to the present invention on silica membrane columns;

FIG. 3 is a tabular summary of the RNA yields and purities determined by UV-VIS (A260, A280), and the Cp value, of an miR-16 qRT-PCR quantification of the eluate diluted 1:10⁵. The long- and short-RNA fractions from porcine liver were recovered in accordance with the method according to the present invention on silica membrane columns;

FIG. 4 shows SDS-PAGE of the proteins precipitated from porcine-liver lysate in accordance with the method according to the present invention. Lane 1: low molecular weight marker; lane 2, 3: 10 μl protein (3 μg/μl); lane 4, 5: 5 μl protein (3 μg/μl);

FIG. 5 is an electropherogram of a fraction of long RNA from HeLa cells, purified in accordance with the method according to the present invention on silica particles;

FIG. 6 is an electropherogram of a short-RNA fraction from HeLa cells, purified in accordance with the method according to the present invention on silica particles;

FIG. 7 is a tabular summary of the RNA yields and purities determined by UV-VIS (A260, A280), and the Cp value, of an miR-16 qRT-PCR quantification of the eluate diluted 1:10⁵. The long- and short-RNA fractions from HeLa cells were recovered in accordance with the method according to the present invention on silica particles;

FIG. 8 shows SDS-PAGE of the proteins precipitated from HeLa-cell lysate in accordance with the method according to the present invention. Lane 1: low molecular weight marker; lane 2, 3: 10 μl protein (0.5 μg/μl);

FIG. 9 shows a 1% TAE agarose gel of a purified in vitro Dicer reaction mixture. Lane 1: 100 by marker; lane 2, 3: 30 μl purified uncut dsRNA; lane 4: 30 μl unpurified reaction mixture; lane 5, 6: 30 μl purified siRNA;

FIG. 10 shows the RNA yield from 30 mg porcine liver without and with prior precipitation of the protein in accordance with the method according to the present invention;

FIG. 11 is an electropherogram of the short-RNA fraction from porcine liver, purified in accordance with the method according to the present invention after protein precipitation with 200 mM Zn;

FIG. 12 is an electropherogram of the short-RNA fraction from porcine liver, purified in accordance with the method according to the present invention after protein precipitation with 400 mM Zn;

FIG. 13 is an electropherogram of the short-RNA fraction from porcine liver, purified in accordance with the method according to the present invention after protein precipitation with 600 mM Zn;

FIG. 14 is a tabular summary of the RNA yields and purities determined by UV-VIS (A260, A280), and the Cp value, of an miR-16 qRT-PCR quantification of the eluate diluted 1:10⁵. The long- and short-RNA fractions from porcine liver were recovered in accordance with the method according to the present invention on silica membrane columns;

FIG. 15 depicts the protein quantities from 30 mg porcine liver quantified in the lysate, after precipitation from the lysate in accordance with the method according to the present invention, and last in the RNA eluate after purification;

FIG. 16 is an electropherogram of the total RNA fraction after purification from 30 mg porcine spleen using AllPrep DNA/RNA/Protein (Qiagen, cat. no. 80004);

FIG. 17 is an electropherogram of the pooled long- and short-RNA fractions after purification from 30 mg porcine spleen in accordance with the method according to the present invention.

### DETAILED DESCRIPTION OF THE PREFERRED EMBODIMENT(S)

### Example 1

### Isolating Protein, Long RNA, and Short RNA from Tissue

### Cell Lysis

30 mg porcine liver was combined with 300 μl lysis buffer (5 M guanidinium thiocyanate, 1% β-mercaptoethanol, 30 mM sodium citrate pH 5) and mechanically comminuted by means of a micropestle until the tissue had almost completely dissolved. The resulting lysate was then separated from unlysed solid constituents by filtration. This was done by centrifuging the lysate through a NucleoSpin® Filter as described in the standard protocol for NucleoSpin® RNA II (kit for isolation of total RNA, Macherey-Nagel, cat. no. 740955.50).

Removing the Long Nucleic Acids

The clear filtrate was adjusted with ethanol (96-100%) to an ethanol concentration of 25%, and carefully mixed in order to establish the binding condition for long nucleic acids. The subsequent steps were carried out in accordance with the NucleoSpin® RNA II protocol (rev. 8, Oct. 2007). A NucleoSpin® RNA II column was loaded with the sample, and the long nucleic acids were bound by centrifuging. This involves a hollow element haying an inlet and outlet opening, into which a silica membrane is introduced as a solid carrier. This was followed by DNase digestion of the DNA bound on the column, and washing and elution of the long RNA remaining on the column, in accordance with the protocol.

Isolating the Protein

The flow through (approx. 400 μl) after the first NucleoSpin® RNA II column was combined with ¾ of a volume (approx. 300 μl) of a protein precipitation buffer (900 mM ZnCl₂, 900 mM sodium acetate pH 5.0) and mixed well. The resulting white, flaky protein precipitate was then isolated by centrifuging (5 min, RT, 14000 g) and removing the supernatant.

Isolating the Small RNA

Tetrahydrofuran (THF) was added to the clear supernatant of the sample after removal of the proteins, so that the THF concentration was approx. 55%. A NucleoSpin® RNA II column in a Collection Tube (2 ml) was then loaded with the sample and centrifuged. In this process, the short nucleic acids bind to the silica carrier (1 min, RT, 11000 g). The column was loaded with 600 μl of a chaotropic salt washing buffer (1.33 M guanidinium thiocyanate, 30 mM sodium citrate pH 7, 66% EtOH) and centrifuged (1 min, 11000 g). This was followed by a second washing step with 600 μl of a chaotropic-salt-free washing buffer (2.5 mM Tris/HCl pH 7.5, 20 mM NaCl, 80% ethanol). The column was then loaded again with 200 μl of this buffer and dried by centrifuging (2 min, RT, 11000 g). The short RNA was then eluted with 50 μl RNase-free water.

Characterizing the Isolated RNA and the Protein

1 μl each of the long and the short RNA eluates were subjected to electrophoresis using an Agilent 2100 Bioanalyzer on an Agilent RNA 6000 Nano Kit chip (cat. no. 5067-1511).

The isolated small- and long-RNA fractions were quantified by measuring absorption at 260 nm (A₂₅₀=1 corresponds to a concentration of approx. 40 μg/ml in a 1 cm standard cuvette). Purity was determined by way of the A₂₆₀/A₂₈₀ quotient.

As a representative of all the isolated miRNAs in the small-RNA fraction, miR-16 was determined in a Roche LightCycler by quantitative RT-PCR per manufacturers instructions (Applied Biosystems cat. no. 4373121, 4324018, and 4366596). The sample was diluted 1:10⁵ for measurement.

The isolated/precipitated proteins were washed and dried in accordance with the NucleoSpin® RNA/Protein standard protocol (Macherey-Nagel, cat. no. 740933.50). The proteins were dissolved in the Protein Loading Buffer (PLB) of the kit and subjected to SDS-PAGE.

Result

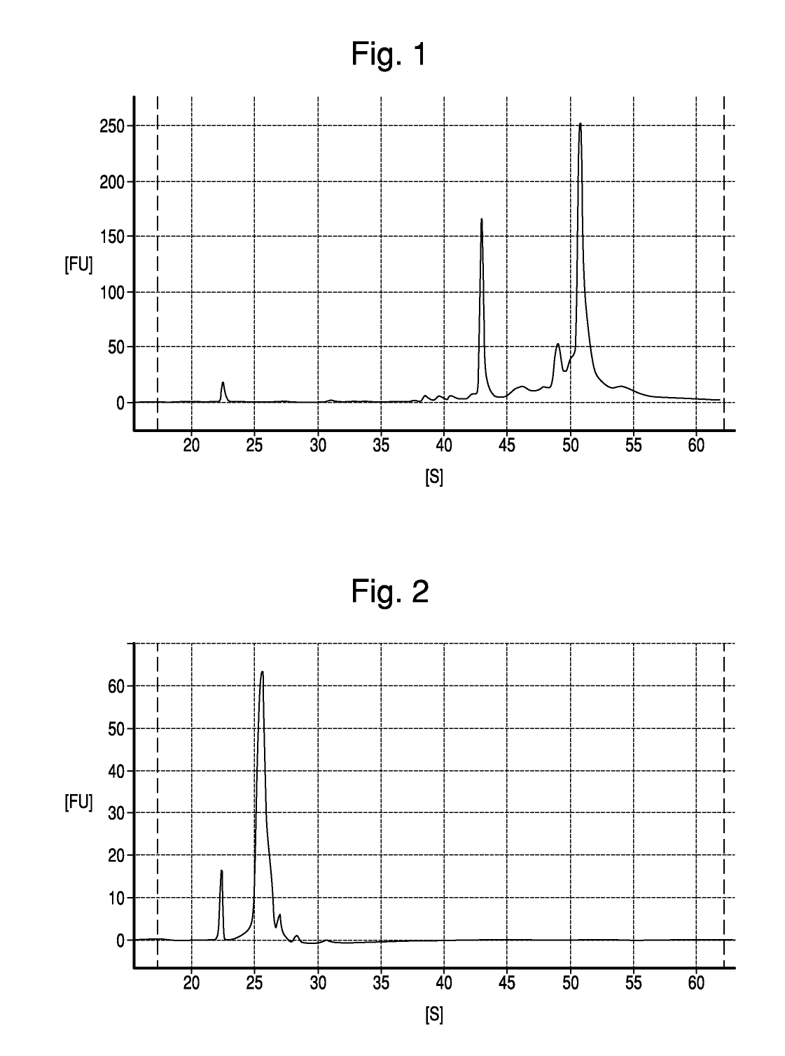

FIG. 1 shows a typical electropherogram of animal RNA, with distinct 18S and 28S rRNA peaks. The short RNAs, which occur in large quantities, are not visible here. They are, conversely, clearly evident in the electropherogram of the purified short-RNA fraction in FIG. 2, at approximately 25 to 30 s just after the marker peak. Residues of long RNA, for example the rRNA peaks that predominate in FIG. 1, are not visible. The two illustrations thus show complete fractionation into short and long RNA.

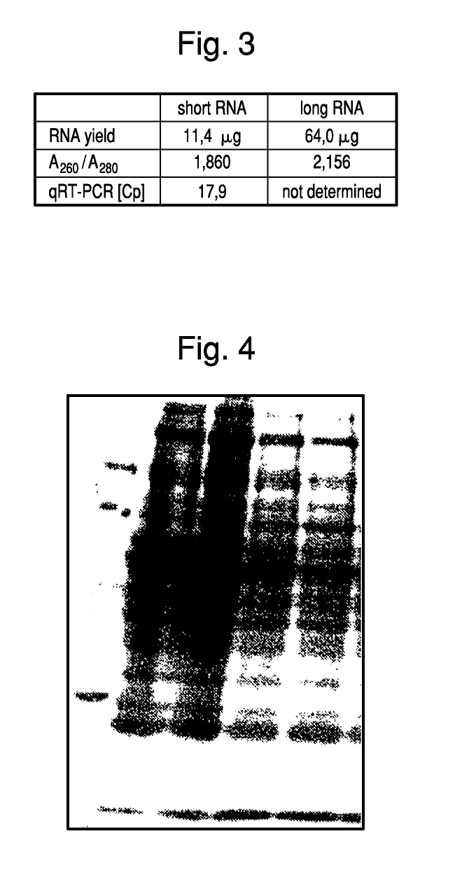

The RNA yields and purities for the two fractions are summarized in FIG. 3. The Cp value of 17.9 in qRT-PCR for the short-RNA fraction diluted 1:10⁵ shows the high concentration of the isolated miRNAs. The negative control of this TagMan® PCR system yields no product even after 50 cycles. The absence of PCR inhibitors was confirmed by dilution series and determination of the amplification efficiency (data not shown).

FIG. 4 shows, with reference to SDS-PAGE, that precipitation did not cause only small or only large proteins to precipitate, but rather that the totality of all cellular proteins, regardless of their size, is visible. This is an important precondition if the proteins are subsequently to be analyzed further (e.g. Western Blot).

### Example 2

### Extracting and Purifying miRNAs from Cells Using Silica Particles

In order to demonstrate the applicability of the method to other sample materials and binding matrices, short and long RNA, as well as protein, was isolated from 3×10⁶ HeLa cells with the aid of silica particles. Lysis and purification were performed as described in Example 1. Instead of the NucleoSpin® RNA II columns with a silica membrane, however, silica particles were used as a binding matrix. The silica particles were suspended in the respective sample or buffer in order to bind the nucleic acids and for the washing and elution steps. Incubation was performed at room temperature for 5 min for binding and elution, and for only 1 min for the washing steps. After centrifuging to sediment the particles, the supernatant was removed and, as applicable, transferred into a new vessel.

Characterization was performed as in Example 1.

Result

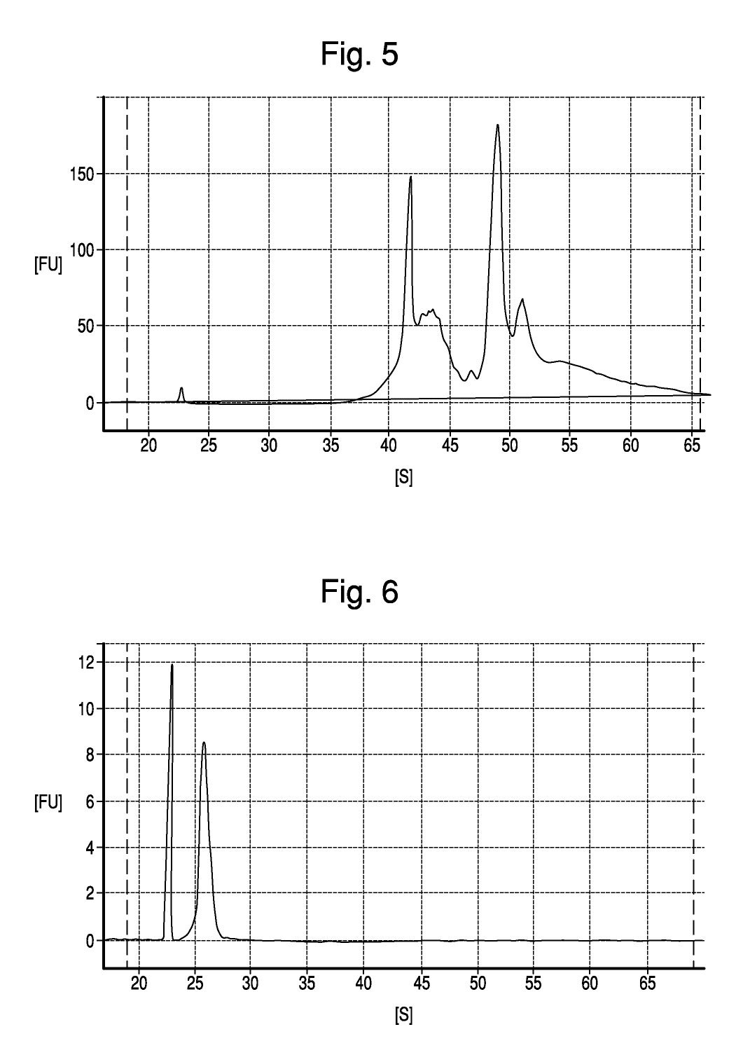

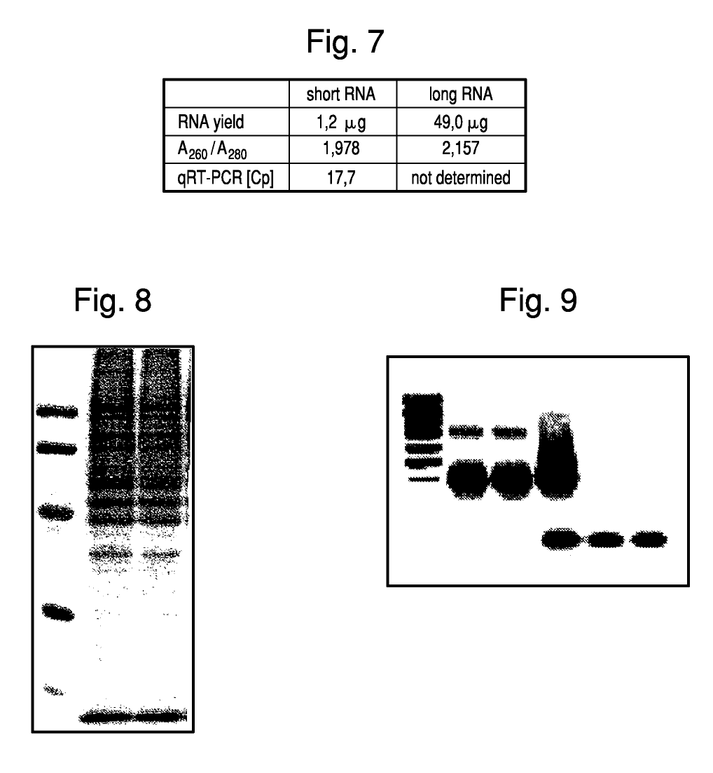

Similarly to Example 1, two fractions were again obtained using silica particles as a solid phase. The long RNA is depicted in FIG. 5, the short in FIG. 6. A tabular summary of the yields and purities is provided in FIG. 7. The protein fraction separated by SDS-PAGE is shown in FIG. 8.

### Example 3

### Purifying siRNAs from an In Vitro Digestion with Dicer

The intention was to completely remove uncut long RNA, as well as the Dicer enzyme, from an in vitro reaction mixture in which long double-stranded RNA (approx. 400 bp) had been cut by the Dicer enzyme into smaller double-stranded siRNA. By varying the quantities of ethanol, lysis buffer, and protein precipitation buffer, the cutoff is shifted downward so that the small siRNA can be cleanly isolated.

Removing the Long dsRNA

150 μl of an in vitro reaction mixture was combined with 150 μl lysis buffer (see Example 1) and 200 μl ethanol (96-100%). A NucleoSpin® RNA II column was loaded with the sample, and the long dsRNA was bound by centrifuging. After binding, the long dsRNA was further purified (washed and eluted) in the same manner as the short RNA of Example 1.

Purifying the siRNA

The flow through was combined with 100 μl protein precipitation buffer, but protein removal was omitted at this point since the reaction mixture contained only minimal quantities of protein. The sample was then combined with 800 μl dioxan and loaded onto a second NucleoSpin® RNA II column. The bound siRNA was then further purified in the same manner as the short RNA of Example 1.

Characterizing the Isolated RNA

The two eluates with the separated long dsRNA and siRNA were analyzed on a 1% TAE EtBr agarose gel (1 h, 75 V).

Result

FIG. 9 shows the successful fractionation of an in vitro reaction mixture (lane 4). Lanes 5 and 6 show clean fractions of the short (21 base pairs) double-stranded siRNA product, which is now completely free of the long (400 base pairs) initial RNA. The latter is depicted, with no residues of the short siRNA, in lanes 2 and 3.

### Example 4

### Purifying Long RNA Before and after Protein Precipitation

This experiment shows that long RNA and DNA are completely precipitated in the context of protein precipitation. It was found, surprisingly, that short RNA remains in solution and can then be purified.

Isolating Long RNA Before Protein Precipitation

Just as in Example 1, three samples were lysed, combined with ethanol, and loaded onto NucleoSpin® RNA II columns. The DNA on the columns was digested as described, the columns were washed, and the long RNA eluted. Precipitation of the protein, and purification of the short RNA out of the filtrate, were omitted.

Isolating Long RNA after Protein Precipitation

Three further samples were likewise lysed and combined with ethanol as described in Example 1. Removal of the long nucleic acids using the NucleoSpin® RNA II column was skipped, however, and the previously described protein precipitation was carried out directly. After removal of the protein precipitate, the clear lysate was adjusted to a final concentration of 55% THF. This concentration of organic solvent is sufficient, as shown in Example 1, to bind even the smallest miRNAs, and hence must also definitely cause the binding of long RNA. The sample was loaded onto a NucleoSpin® RNA II column and the nucleic acids were bound by centrifuging (1 min, RT, 11000 g). Like the first three samples of this exemplifying embodiment, the bound nucleic acids were subjected to a DNase digestion operation and then washed and eluted.

Quantifying the Long RNA

All six eluates were quantified by absorption measurement at 260 nm, as described in Example 1.

Result

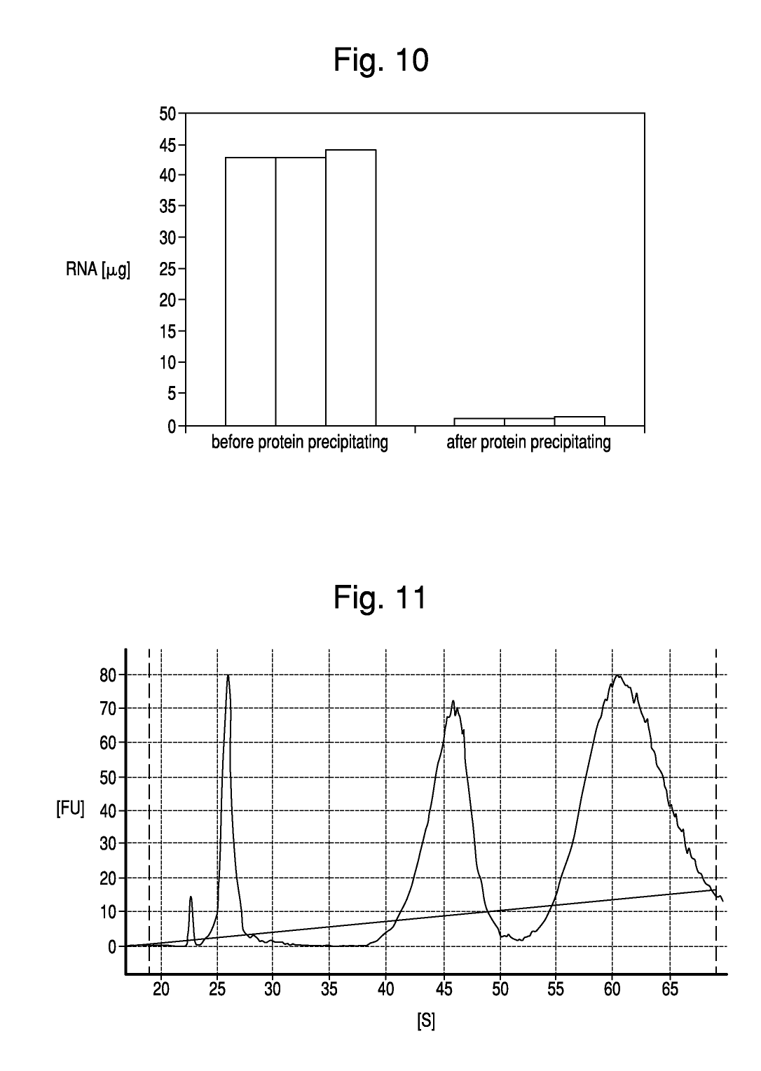

As FIG. 10 clearly shows, the long RNAs are also precipitated, almost quantitatively, in the course of protein precipitation, Surprisingly, however, the short RNAs remain in solution and can subsequently be purified as shown in Example 1.

### Example 5

Extracting and Purifying RNAs after Protein Precipitation at Variable Zn Concentration

Example 4 showed that long RNA is precipitated together with the protein by zinc, but short RNA remains in solution. This Example now demonstrates more specifically the correlation between the zinc concentration and the RNA lengths thereby precipitated.

Purifying Short RNA

Example 1 was repeated exactly, except that the quantity of zinc in the protein precipitation buffer was adjusted so that it yielded a final concentration of 200, 400, or 600 mM after addition to the lysate.

Characterizing the Isolated RNA

As described in Example 1, the eluates were analyzed using an Agilent 2100 Bioanalyzer and quantified by UV-VIS and qRT-PCR.

Result

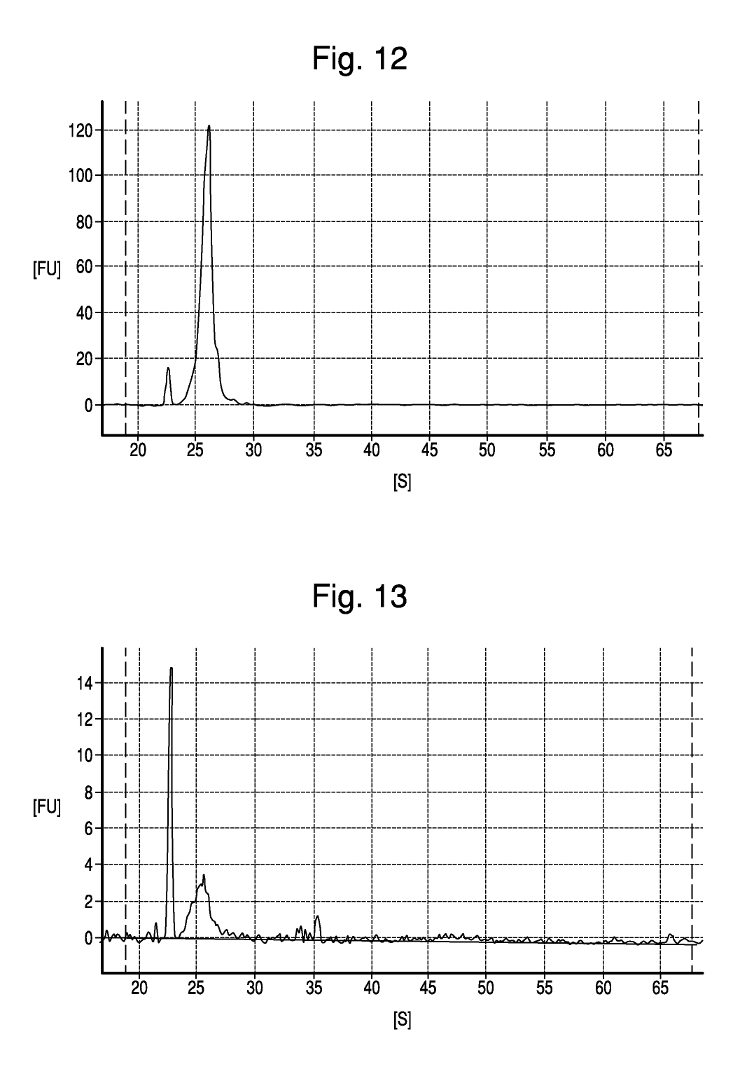

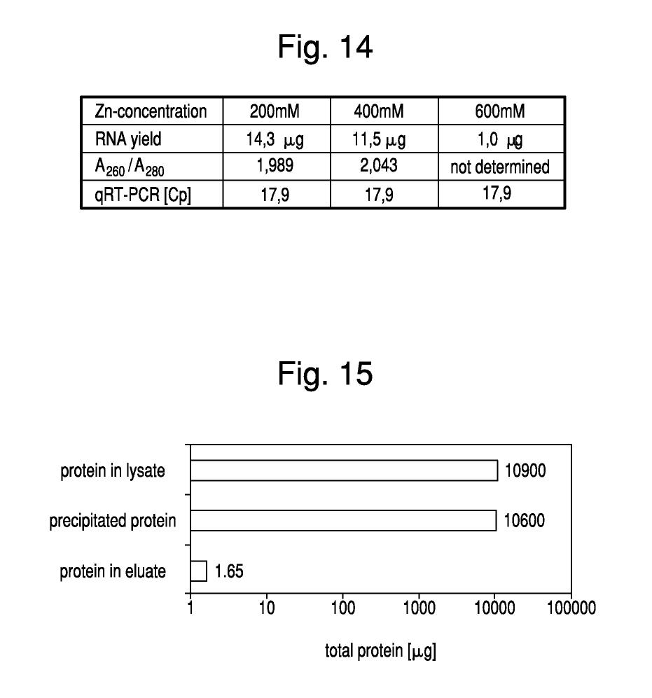

As shown by FIGS. 11, 12, and 13, isolation of the short RNAs follows an optimum curve for zinc concentration. As the zinc concentration increases, shorter nucleic acid fractions are also increasingly co-precipitated in the course of protein precipitation. in FIG. 11, for example, with 200 mM zinc, in addition to the small RNAs at approx. 25 s, wide peaks for genomic DNA are also visible. The genomic DNA is already absent at 400 mM (FIG. 12), an indication that under these conditions, the long nucleic acids are precipitating out with the protein. When the zinc is increased to 600 mM (FIG. 13), the yield of the short-RNA fraction also sharply decreases. This especially affects RNA species such as tRNA or 5.8S rRNA, which are still visible in the electropherogram at the short retention times. The miRNAs, on the other hand, are not affected, and do not precipitate even with 600 mM zinc, as shown by the quantification using qRT-PCR in FIG. 14. Identical miRNA concentrations (evident from the identical Cp value) were identified regardless of the zinc concentration.

### Example 6

### Protein Balance

This exemplifying embodiment confirms that the proteins are essentially completely removed by precipitation with zinc.

Isolating the Protein and the Short RNA

60 mg porcine liver was lysed in 600 μl lysis buffer and clarified, as described in Example 1. The protein and the short RNA were recovered from 300 μl lysate, as described.

Protein Determination

The precipitated protein was redissolved in 300 μl lysis buffer and subjected, together with unprecipitated lysate, to a protein determination. For this, both samples were diluted 1:100 in lysis buffer, and a calibration curve in lysis buffer was prepared for the Micro BCA Protein Assay Reagent kit (Pierce, cat. no. 23235) that was used.

In the same mixture, the residual protein in the short-RNA fraction was determined against a calibration curve in water.

Result

For 300 μl initial lysate, the result was a protein concentration of 36.3 μg/μl and thus a total protein quantity of approx. 10.9 mg from 30 mg of tissue (FIG. 15).

For the precipitated and redissolved protein, the result was a concentration of 35.2 μg/μl in 300 μl, and therefore a total protein quantity of 10.6 mg.

The protein content in the short-RNA fraction was only 0.033 μg/μl in 50 μl eluate, and thus a total protein content of 1.65 μg.

The concentration had therefore been reduced by a factor of approximately 1000, and the absolute quantity by a factor of approx. 6600. Referred to the initial lysate, precipitation thus removed 97% of the protein and made it accessible to further analysis.

### Example 7

### Comparative Example

### Depletion of Short RNA by the Qiagen AllPrep DNA/RNA/Protein Kit

This exemplifying embodiment shows that in a context of simultaneous purification of RNA and protein from a sample according to the existing art, the short RNAs are lost, with a cutoff of approx. 200 bases.

Isolating the RNA

RNA was obtained from 30 mg porcine spleen in accordance with the manufacturer's protocol for the AllPrep DNA/RNA/Protein Kit (Qiagen, cat. no. 80004), and eluted in 100 μl buffer.

Concurrently therewith, a 50 μl long-RNA fraction and a 50 μl short-RNA fraction from 30 mg porcine spleen were isolated in accordance with the method according to the present invention and as described in Example 1, and then pooled.

Characterizing the RNA

Both samples were investigated using an Agilent Bioanalyzer, as described in Example 1.

Result

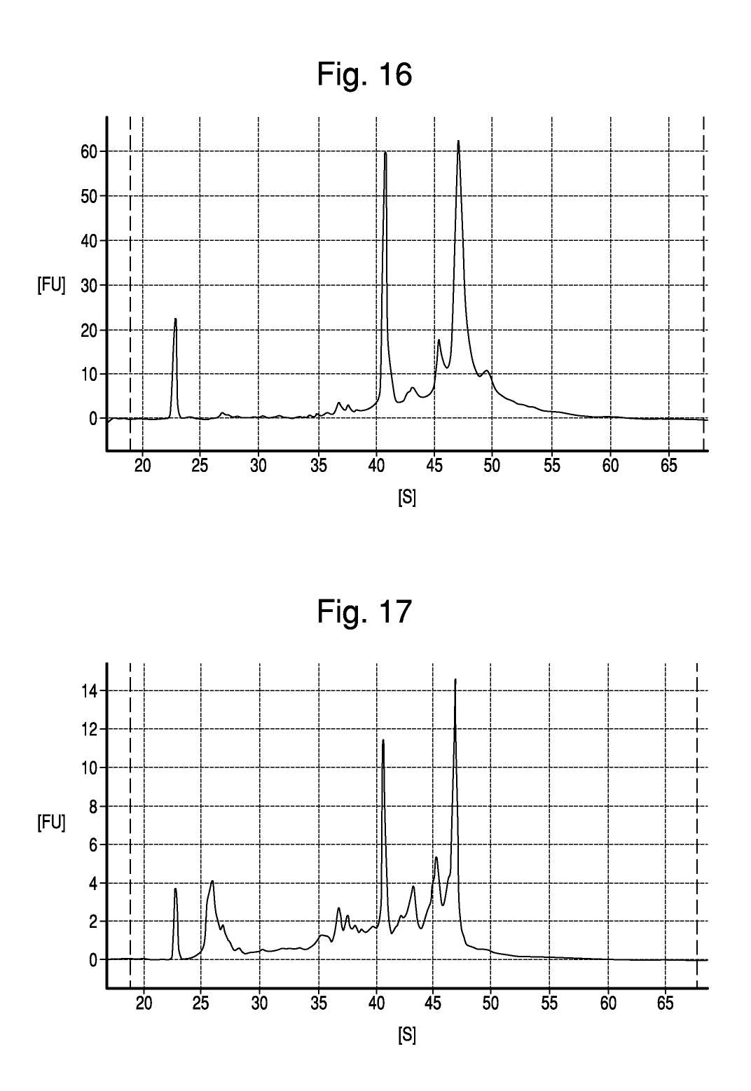

FIG. 16 shows clearly that with the Qiagen All/Prep DNA/RNA/Protein kit, almost all the RNA in the range up to 200 bases is lost (peaks at approx. 25 s absent or small), while the method according to the present invention purifies out short RNAs almost quantitatively (large peak for short RNAs at approx. 25 s, FIG. 17).

We claim:

1. A method for recovering at least short RNA, having at least the following steps:
a) making available a biological solution containing at least short RNA as well as proteins and/or long nucleic acids (long RNA and DNA);
b) removing the proteins and the long nucleic acids from the solution, at least the proteins being precipitated;
c) adsorbing the short RNA onto a solid (first) carrier after precipitation of the proteins;
d) recovering the short RNA by desorption from the carrier.

2. The method according to claim 1, wherein the proteins and the long nucleic acids are separated from one another, and the proteins and/or the long RNA is/are recovered.

3. The method according to claim 1, wherein the long nucleic acids are removed from the solution before or concurrently with precipitation of the proteins.

4. The method according to claim 2, wherein the long nucleic acids are adsorbed onto a solid (second) carrier.

5. The method according to claim 4, wherein the solution for adsorption of the long nucleic acids has an organic solvent, in particular water-miscible solvent added to it, in a concentration such that only the long nucleic acids adsorb on the second carrier, the concentration of the organic solvent being adjusted in particular to a range from 15 to 40 vol %, by preference 20 to 30 vol %, particularly preferably 25 vol %, based in each case on the solution with the solvent(s) added to it.

6. The method according to claim 4, wherein the solution for adsorption of the long nucleic acids has added to it a salt of high ionic strength, in particular a chaotropic salt or multiple chaotropic salts, the concentration of which in the solution is adjusted in particular to a range from 1 to 10 M.

7. The method according to claim 4, wherein DNA is removed, in particular by DNase digestion, from the nucleic acids adsorbed onto the second carrier, and the remaining long RNA is isolated, especially is eluted from the second carrier, optionally after a washing operation.

8. The method according to claim 1, wherein the solution for precipitation of the proteins has metal ions added to it, in particular divalent metal ions, for example from the group of the elements Fe, Co, Ni, Cu, Zn, Cd, Hg, Pb, and Ba, the concentration of the metal ions being adjusted in particular to at least 0.01 M to a maximum of 1.5 M, by preference 0.05 to 1 M, even better 0.1 to 0.8 M, particularly preferably 0.2 to 0.6 M, and optimally 0.3 to 0.4 M.

9. The method according to claim 1, wherein for adsorption of the short RNA, a solution is used that contains an organic solvent at high concentration, the concentration being adjusted in particular to a value from 30 to 80 vol %, by preference 40 to 70 vol %, particularly preferably 50 to 60 vol %, based in each case on the solution with the solvent added to it; the organic solvent preferable being a nonalcoholic, water-miscible solvent, which is selected, for example, from the group that includes acetone, acetonitrile, dimethyl sulfoxide (DMSO), tetrahydrofuran (THF), dioxan, and dimethylformamide, is used as an organic solvent.

10. A kit for recovering at least short RNA, comprising:
a) at least one solid carrier for the adsorption of nucleic acids;
b) a protein-precipitating reagent;
c) at least one binding substance for selective adsorption of short RNA on a solid carrier;
d) instructions having the method steps according to one of claims 1 to 9.

11. The kit according to claim 10, wherein at least one binding substance for adjusting the binding conditions for selective adsorption of long nucleic acids on a solid carrier, in particular a water-miscible organic solvent and/or a salt from among one or more chaotropic salts, is present as at least one binding substance, and/or the kit comprises a DNase for DNA digestion.

12. The kit according to claim 10, wherein a protein-precipitating reagent containing metal ions is present in the kit, the metal ions being made up of the group of elements to which Fe, Co, Ni, Cu, Zn, Cd, Hg, Pb, and Ba belong.

13. The kit according to claim 10, wherein an organic solvent, in particular a solvent that is water-miscible and nonalcoholic, is present as at least one binding substance for the adsorption of short RNA, and is selected e.g. from the group to which acetone, acetonitrile, dimethyl sulfoxide (DMSO), tetrahydrofuran (THF), dioxan, and dimethylformamide (DMF) belong.

14. The kit according to claim 10, wherein particles, including in the form of powders or suspensions, polymers, membranes, filter layers, frits, nonwoven fabrics, or carriers in the form of a monolith, are present as solid carriers, and/or the material for the solid carrier is silica, glass, quartz, zeolites, or mixtures thereof, and/or magnetic particles preferably coated with silica, glass, quartz, or zeolites.

15. The kit according to claim 10, wherein at least one elution reagent, for desorption of at least the short RNA from the carrier, and/or at least one washing buffer is present.
